(19) United States
(12) Patent Application Publication
(10) Pub. No.: US 2003/0092603 A1
(43) Pub. Date: May 15, 2003

(54) Identification of specific modulators of bone formation

(76) Inventors: Gregory R. Mundy, San Antonio, TX (US); I. Ross Garrett, San Antonio, TX (US); Di Chen, San Antonio, TX (US); G. Rossini, San Antonio, TX (US); M. Zhao, Helotes, TX (US)

Correspondence Address: Kate H. Murashige, Morrison & Foerster LLP, Suite 500, 3811 Valley Centre Drive, San Diego, CA, 92130-2332, US

(21) Appl. No.: 10/268,374

(22) Filed: Oct. 9, 2002

Related U.S. Application Data
(60) Provisional application No. 60/328,300, filed on Oct. 9, 2001.
(60) Provisional application No. 60/346,742, filed on Jan. 7, 2002.

(51) Int. Cl. A61K 31/00; C12Q 1/68; G01N 33/567

(52) U.S. Cl. 514/1; 435/6; 435/7.21

(57) ABSTRACT

The E3 ubiquitin ligases which are specific to ubiquitination of proteins relevant to bone formation are useful targets for protocols or compounds to ameliorate bond disorders. These ligases are β-TrCP, Smurf1 and Smurf2.

### CROSS-REFERENCE TO RELATED APPLICATIONS

This application claims benefit under 35 U.S.C. § 119(e) of U.S. application Ser. No. 60/346,742 filed Jan. 7, 2002 and U.S. Ser. No. 60/328,300 filed Oct. 9, 2001. The contents of these applications are incorporated herein by reference.

### TECHNICAL FIELD

The invention relates to the metabolic events associated with bone formation and the use of specific medicaments to regulate this metabolism. More specifically, it concerns the use of compounds that inhibit ubiquitin E3 ligases that control the levels of essential transcription factors and signaling proteins in bone formation.

### BACKGROUND ART

It has been established that inhibitors of proteasome activity have the effect of enhancing bone formation through, at least in part, enhanced differentiation of osteoblasts, the cells which are responsible for bone formation. It us understood that skeletal elements, and in particular bone, are subject to constant breakdown and resynthesis wherein osteoblasts produce new bone, while osteoclasts destroy it. The differentiation of progenitors into osteoblasts and osteoclasts and regulation of the activities of the resulting cells are accomplished in a complex process incompletely understood. It is, however, known that certain proteins, known as bone morphogenic proteins (BMPs) relate to the overall process. Of particular importance is BMP-2, one of the several BMPs understood to be relevant.

As stated above, it is known that inhibitors of proteasomal activity promote bone formation and hair growth. PCT publication WO 00/2548, published Jan. 20, 2000 and incorporated herein by reference provides data which demonstrate that certain inhibitors of proteasomal activity stimulate the formation of bone and activate the expression of BMP-2. Among these inhibitors are PSI and MG-132, which are peptide aldehydes. A subsequently published PCT application, publication No. WO 01/28579 published Apr. 26, 2001, also incorporated herein by reference, discloses additional classes of compounds known to be proteasome inhibitors that enhance production of BMP-2 and bone formation. These compounds include epoxides such as epoxomicin, boronates, and sulfonates. These compounds are believed to react with the threonine residue of the chymotrypsin-like catalytic site of the proteasome.

A helpful review of the nature of the proteasome and classes of inhibitors of its activity is found, for example, in an article by Adams, J., et al., Ann. Reports in Med. Chem. (1996) pages 279-288, also incorporated herein by reference. Briefly, the proteasome is believed to comprise a 20S catalytic structure described as a stack of four oligomeric rings forming a core through which a protein to be degraded must pass. The passage of the protein through the core for degradation is controlled by two copies of a 19S regulatory complex, one copy at each end, to result in the 26S proteasome. At the time of this review, nine different hydrolytic activities had been described for the proteasome, but the three major such activities include a chymotrypsin-like activity, a trypsin-like activity and a peptidyl glutamyl peptide hydrolyzing (PGPH) activity. As stated above, it appears that inhibitors of the proteasome which inhibit the chymotrypsin-like activity are effective in enhancing bone formation.

Any protein that enters the proteasome to be degraded must first be ubiquitinated. Ubiquitin is an evolutionarily conserved protein found in all eukaryotic cells and contains 76 amino acid residues. The C-terminal glycine of ubiquitin is first ligated onto an ε amino group of any lysine residues of the targeted protein, and then the lysine at position 48 of ubiquitin is coupled to the carboxy terminus of another molecule of ubiquitin and subsequent rounds of ubiquitination ensue. The ubiquitinated proteins can then enter the proteasome for degradation.

Protein ubiquitination involves a cascade of enzymatic reactions catalyzed by the E1 ubiquitin-activating enzyme, the E2 ubiquitin-conjugating enzymes, and the E3 ubiquitin ligases. The E3 ubiquitin ligases play a crucial role in defining substrate specificity and subsequent protein degradation by the 26S proteasomes. Two major types of E3 ligases are Hect domain E3 ligases and SCF-domain E3 ligases.

Hect domain ligases contain a conserved cysteine, located at the carboxy terminus of the Hect domain, which forms a thioester bond with ubiquitin transferred from an appropriate E2 enzyme. This E3-ubiquitin thioester is then the donor for amide bond formation with the protein to be degraded. Another motif often found in the Hect family of E3 ligase is the WW domain, which contains two highly conserved tryptophans and a conserved proline in an approximately 30-amino acid region. The WW domains have a preference for binding to small proline-rich sequences, PPXY motifs, and different WW domains possess different substrate specificity. Zhu, H., et al., Nature (1999) 400:687-693.

One E3 ligase of particular importance to the present invention is the E3 ubiquitin ligase β-TrCP, which is specific for the processing of a transcription factor designated Gli3. Gli3 is the mammalian counterpart of the drosophila transcription factor cubitius interruptus (ci) which regulates the expression of decapentaplegic (dpp) the homolog of the BMP-2 and BMP-4 genes. It has been shown that ci is a powerful transcriptional activator of dpp. See Aza-Blanc, P., et al., Cell (1997) 89:1043-1053 and Methot, N., et al, Cell (1999) 96:819-831. On the other hand, enzymatic processing of ci by the ubiquitin proteasome pathway results in a cleaved form which is a repressor of dpp expression. See Jiang, J., et al., Nature (1998) 391:493-496; Ingham, P. W., Embo. J. (1998) 17:3505-3511; Chen, C. H., et al., Cell (1999) 98:305-316.

Like ci, Gli3 is known to be processed by the ubiquitin proteasome pathway to produce a truncated form with deletion of the C-terminus. See Dai, P., et al., J. Biol. Chem. (1999) 274:8143-8152. Humans with naturally occurring mutations in Gli3, and animals with Gli3 gene deletion show abnormal skeletal development and the absence of the Gli3 gene results in activation of BMP gene expression. See Theil, T., et al., Cell Tissue Res. (1999) 296:75-83.

It has now been found that inhibition of the Gli3-specific E3 ubiquitin ligase, an SCF domain ligase, β-TrCP, results in enhancement of bone formation, and thus inhibitors of this enzyme are useful in the treatment of various conditions that are characterized by bone defects. Accordingly, assays for inhibitors of β-TrCP and its counterparts in other related species result in identification of compounds specifically focused on treatment of these bone disorders.

Other E3 ligases relevant to this invention include Smurf1 and Smurf2. Smurf1 and 2 (Smad ubiquitin regulatory factor 1 and 2) are members of the Hect family of E3 ligase and interact with the BMP-activated Smads 1 and 5, thereby triggering their ubiquitination and degradation. Smads1 and 5 are immediate downstream signaling molecules of BMP receptor and play a central role in BMP signaling. A summary of this sequence of events is found, for example, in an article by Zhu, H., et al., Nature (1999) 400:687-693; Lin, X., et al., J Biol Chem (2000) 275:36818-36822; Kavsak, P., et al., Mol Cell (2000) 6:1365-1375; Zhang, Y., et al., Proc. Natl. Acad. Sci. USA (2001) 98:974-979. Briefly, binding of BMP ligands to their receptors results in phosphorylation of the intracellular GS domain of the type I BMP receptor by a type II receptor kinase. The intracellular kinase domain of the type I receptor phosphorylates specific Smad proteins, Smads 1, 5 and 8. In vertebrates, these phosphorylated Smads associate with a member of a second class of Smad, the common mediator Smad4. The associated Smad4/Smad 1, 5, or 8 complex, translocates into nucleus and possibly in association with an additional transcription factor, regulates transcription of one or more target genes in the nucleus. See, for example, Helden, C -H., et al., Nature Cell. Biol. (1989) 1:E195-E197. An additional Smad, Smad6, is relevant to BMP signaling, as it inhibits signaling by BMP.

The WW domain of Smurf1 interacts with the PY motif in the linker region of Smads 1 and 5 but not with Smads which respond to activin or TGFβ receptors(Zhu, et al., supra.). Zhang, Y., et al. supra, also report that Smurf2 preferentially targets Smad1 in preference to either Smad2 or Smad3, which respond to TGFβ and activin.

Recent studies by applicants have shown that in addition to mediating Smad1 and 5 degradation, Smurf1 also interacts with Cbfa1 protein, a critical transcription factor in controlling bone development and osteoblast function, and mediates Cbfa1 degradation in an ubiquitin-proteasome-dependent manner (Zhao, et al., 2002).

Thus, the effects of BMP signaling and BMP transcription are controlled by at least three E3 ubiquitin ligases, β-TrCP, Smurf1 and Smurf2 whose activities result in interrupting the normal course of osteoblast differentiation and/or activity.

Abstracts relating to the activity of Smurf1 were submitted to the American Society for Bone and Mineral Research (ASBMR) 2001 and 2002 annual meetings by applicants. A manuscript describing the effect of Smurf1 on Cbfa1 degradation and osteoblast differentiation has been submitted to Journal of Cell Biology for publication by applicants.

### DISCLOSURE OF THE INVENTION

The invention is directed to methods to identify compounds and protocols which will be effective in stimulating bone formation and/or differentiation of osteoblasts by identifying specific inhibitors for E3 ubiquitin ligases that mediate Gli3 processing or Smad1 and 5 degradation. Inhibition of these ligases, β-TrCP, Smurf1 and Smurf2, promotes the differentiation of osteoblasts and the formation of new bone. Thus, by identifying inhibitors of these enzymes, compounds highly focused on the metabolic events that result in the formation of bone may be found.

Thus, in one aspect, the invention is directed to a method to identify a compound or protocol which enhances bone formation and/or osteoblast differentiation which method comprises assessing the ability of a candidate compound or protocol to inhibit the activity of β-TrCP, whereby a compound or protocol which inhibits this activity is identified as a compound or protocol which will enhance bone formation and/or osteoblast differentiation.

In another aspect, the invention is directed to a method to identify a compound or protocol which enhances bone formation and/or osteoblast differentiation which method comprises assessing the ability of a candidate compound or protocol to inhibit the activity of Smurf1 or Smurf2, whereby a compound or protocol which inhibits this activity is identified as a compound or protocol which will enhance bone formation and/or osteoblast differentiation.

In other aspects, the invention is directed to methods to enhance bone formation and/or osteoblast differentiation or proliferation by administering to a subject in need of such treatment compounds or protocols identified by the above methods. In another aspect, the invention is directed to methods to treat various bone defect conditions which method comprises administering to a subject in need of such treatment an effective amount of a compound or of a protocol which is inhibitory to β-TrCP, Smurf1 and/or Smurf2.

### BRIEF DESCRIPTION OF THE DRAWINGS

FIG. 1 is a graph showing the correlation of bone formation enhancement with inhibition of chymotrypsin activity.

FIGS. 2a and 2b are photocopies of gels which show the effect of epoxomicin on degradation of Flag-tagged Gli3 in 293 cells and C3H10T1/2 cells, respectively.

FIG. 2c is a photocopy of a gel which shows the effect of β-TrCP (labeled Slimb in the Figure) on Gli3 degradation.

FIG. 2d is a graph showing the effect of β-TrCP on BMP-2 promoter activity in the presence and absence of Gli3.

FIGS. 3a and 3b show the effect of truncated Gli3 on BMP-2 promoter activity in the presence and absence of PS-1.

FIGS. 4a and 4b show the effect of Smad1 and mutant Smurf1 on the production of alkaline phosphatase and osteocalcin in the presence and absence of BMP-2.

FIGS. 5a and 5b are photocopies of gel which show the effect of Smurf1 and mutant Smurf1 on the levels of F-Smad1.

FIGS. 5c and 5d are photocopies of gel which show the effect of adding various proteasome inhibitors on the ability of Smurf1 to decrease the levels of F-Smad1.

FIG. 6a is a photocopy of a gel which shows the ability of Smurf1 to decrease the levels of the protein Cbfa.

FIG. 6b shows a ubiquitin ladder obtained when ubiquitin is added to cells transfected with Smurf1 and Cbfa1.

FIG. 6c photocopy of a gel displaying the effect of PS-1 on the ability of Smurf1 to deplete Cbfa1.

FIG. 6d is a photocopy of a gel which shows immunoprecipitation of a mutant form of Smurf1 with Cbfa.

FIG. 6e is a graph showing the effects of Smurf1 to modulate the function of Cbfa1.

### MODES OF CARRYING OUT THE INVENTION

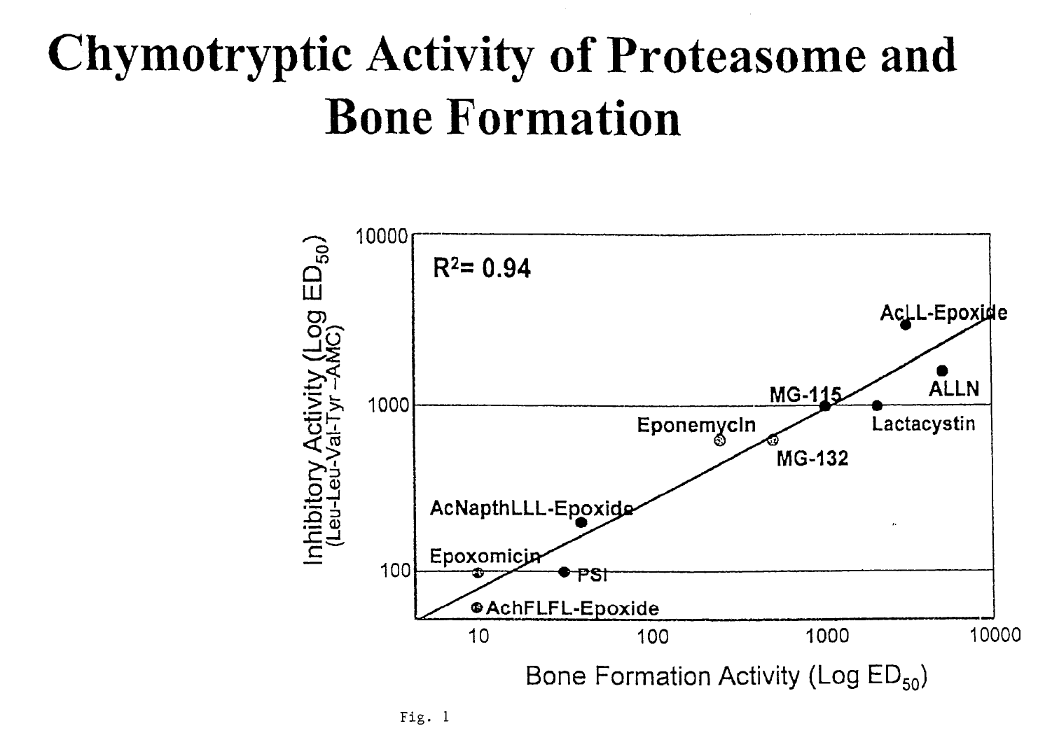

In earlier PCT published applications, set forth hereinabove and incorporated herein by reference, it has been established that inhibitors of proteasome activity, and specifically of the chymotrypsin type of proteasome activity are successful in enhancing the formation of bone in vertebrate subjects. Indeed, as shown in FIG. 1, the ability of known proteasome inhibitors to enhance bone formation is highly positively correlated with the ability of the inhibitor to inhibit the activity of enzymes, which have chymotrypsin activity. However, as the proteasome has only three major types of proteolytic activity, it is evident that even the chymotrypsin activity specifically will be relevant to a multiplicity of pathways affecting the metabolism of the cell, not just those related to bone. It would be helpful to evaluate inhibitors based on their effects on enzymes which are related more uniquely to pathways which are directed to maintenance of adequate bone structure.

Three such targets are described herein. The first is β-TrCP, an E3 ligase, which is specific for the transcription factor Gli3 of which processed short form inhibits BMP-2 expression. β-TrCP is a known protein and the nucleotide sequences encoding it in humans and mice are known. Murine β-TrCP (also called FWD1) cDNA can be obtained by amplifying template RNA extracted from 2T3 cells by reverse transcription PCR. The nucleotide sequence can be verified as compared to deposited GenBank sequence. The β-TrCP protein from any species may be used as may any functional equivalent. Preferably, to identify compounds for use in treating humans, the human form of β-TrCP is used in the assay. As stated above, inhibition of β-TrCP activity results in reducing the production of short form of Gli3 which inhibits BMP-2 gene transcription and expression in osteoblasts.

The other two E3 ligases, which are useful in the invention are Smurf1 and Smurf2. As stated above, these enzymes regulate the levels of Smads 1 and 5, which are essential molecules of the signaling pathway in response to ligand binding to the BMP-2 receptor. Smurf1 is a known protein whose gene has been cloned and sequenced (Zhu, H., et al., Nature (1999) 400:687-693). Smurf1 has a conserved cysteine located at the carboxyl end of the Hect domain, which forms a thioester bond with ubiquitin (Huibregise, J. M., et al., Proc. Natl. Acad. Sci. USA (1995) 92:2563-2567). As stated above, it contains a WW domain, which has a preference for binding to the region containing small proline-rich sequences, PPXY motifs, of Smad1 and 5. Smurf2 is also a previously isolated and sequenced protein. Both human and Xenopus Smurf2 sequences are deposited at GenBank database, accession numbers AY14180 and AY01481 respectively. Human Smurf2 was isolated as described in Zhang, Y., et al., PNAS (2001) 98:974-979, incorporated herein by reference and cited herein above. Thus, both Smurf1 and Smurf2 sequences are available in the art. In addition, for the assays, that portion of Smurf1 and Smurf2, which constitutes the binding region may be substituted for the full-length protein in some instances.

Any of β-TrCP, Smurf1 or Smurf2 can be used in the assays of the invention to identify inhibitors of their activity and thereby stimulators of bone formation. The human or murine forms of these ligases could be used, or the corresponding ligases derived from other species could be substituted in these assays provided sufficient cross-species reactivity is exhibited. Also, minor changes in primary structure can often be tolerated so long as the activity and specificity of the proteins are maintained. Preferably, to identify compounds for use in human therapeutics or prophylactics, the human forms of these ligases will be used in the assays. Alternatively, for veterinary use, it may be preferable to use the ligase derived from the species to be treated. With respect to any variants, it is necessary to verify that the interaction is maintained with the factors that are substrates for the enzyme activity. Design and performance of assays for inhibition of the activity of these enzymes is well within ordinary skill. For example, in one approach, the reaction mixture would contain the relevant E3 ligase (β-TrCP, Smurf1 or Smurf2) ubiquitin and the substrate appropriate for the ligase. The activity of the enzyme can be assessed by measuring the decrease in concentration of ubiquitin, the decrease in concentration of the substrate, or the increase in ubiquitinated product. Alternatively, proteasomes can be added to the reaction mixture and the level of cleaved substrate assessed. Means for assessing these components of the reaction mixture include, without limitation, chromatographic methods or utilization of labels, including generation of colored or fluorescent products.

Once the assay for activity is established, the effect of particular compounds or protocols on the activity can be assessed by conducting the assay in the presence and absence of the protocol or compound and determining whether the activity as measured in the assay increases or decreases or remains the same. Compounds or protocols, which decrease the activity as determined in the assay systems, are then identified as suitable compounds or protocols for enhancing bone growth.

For example, to assess the activity of β-TrCP, Gli3 would be used as substrate in combination with ubiquitin. The rate of ubiquitination can then be measured conveniently by Ross will provide the assay protocol. If proteasomes are added to the assay, the relative amounts of Gli3 precursor protein and processed form of Gli3 can be detected using suitable antibodies. A wide various of methods for such assays can be imagined and devised by the ordinarily skilled artisan.

The components of these assays may be packaged into “kits” and appropriate instructions included.

The identified protocols or compounds are then useful in treating subjects in need of bone formation enhancement. These methods of treatment will, of course, depend on the nature of the compounds and protocols identified.

The ultimate goal of the methods and compositions of the invention is to treat or ameliorate bone disorders in vertebrate subjects, particularly mammals, and more particularly humans.

As used herein, “treat” or “treatment” include a postponement of development of bone deficit symptoms and/or a reduction in the severity of such symptoms that will or are expected to develop. The terms further include ameliorating existing bone or cartilage deficit symptoms, preventing additional symptoms, ameliorating or preventing the underlying metabolic causes of symptoms, preventing or reversing bone resorption and/or encouraging bone growth. Thus, the terms denote that a beneficial result has been conferred on a vertebrate subject with a cartilage, bone or skeletal deficit, or with the potential to develop such deficit.

By “bone deficit” is meant an imbalance in the ratio of bone formation to bone resorption, such that, if unmodified, the subject will exhibit less bone than desirable, or the subject's bones will be less intact and coherent than desired. Bone deficit may also result from fracture, from surgical intervention or from dental or periodontal disease. By “cartilage defect” is meant damaged cartilage, less cartilage than desired, or cartilage that is less intact and coherent than desired. “Bone disorders” includes both bone deficits and cartilage defects.

Representative uses of the compounds identified by the method of the present invention include: repair of bone defects and deficiencies, such as those occurring in closed, open and non-union fractures; prophylactic use in closed and open fracture reduction; promotion of bone healing in plastic surgery; stimulation of bone in-growth into non-cemented prosthetic joints and dental implants; elevation of peak bone mass in pre-menopausal women; treatment of growth deficiencies; treatment of periodontal disease and defects, and other tooth repair processes; increase in bone formation during distraction osteogenesis; and treatment of other skeletal disorders, such as age-related osteoporosis, post-menopausal osteoporosis, glucocorticoid-induced osteoporosis or disuse osteoporosis and arthritis, or any condition that benefits from stimulation of bone formation. The compounds identified by the method of the present invention can also be useful in repair of congenital, trauma-induced or surgical resection of bone (for instance, for cancer treatment), and in cosmetic surgery. Further, these can be used for limiting or treating cartilage defects or disorders, and may be useful in wound healing or tissue repair.

The compounds identified by the method of the invention may be administered systemically or locally. For systemic use, the compounds herein are formulated for parenteral (e.g., intravenous, subcutaneous, intramuscular, intraperitoneal, intranasal or transdermal) or enteral (e.g., oral or rectal) delivery according to conventional methods. Intravenous administration can be by a series of injections or by continuous infusion over an extended period. Administration by injection or other routes of discretely spaced administration can be performed at intervals ranging from weekly to once to three times daily. Alternatively, the compounds may be administered in a cyclical manner (administration of compound; followed by no administration; followed by administration of compound, and the like). Treatment will continue until the desired outcome is achieved. In general, pharmaceutical formulations will include an active ingredient identified by the methods herein in combination with a pharmaceutically acceptable vehicle, such as saline, buffered saline, 5% dextrose in water, borate-buffered saline containing trace metals or the like. Formulations may further include one or more excipients, preservatives, solubilizers, buffering agents, albumin to prevent protein loss on vial surfaces, lubricants, fillers, stabilizers, etc. Methods of formulation are well known in the art and are disclosed, for example, in Remington's Pharmaceutical Sciences, latest edition, Mack Publishing Co., Easton Pa., which is incorporated herein by reference.

Pharmaceutical compositions for use within the present invention can be in the form of sterile, non-pyrogenic liquid solutions or suspensions, coated capsules, suppositories, lyophilized powders, transdermal patches or other forms known in the art. Local administration may be by injection at the site of injury or defect, or by insertion or attachment of a solid carrier at the site, or by direct, topical application of a viscous liquid, or the like. For local administration, the delivery vehicle preferably provides a matrix for the growing bone or cartilage, and more preferably is a vehicle that can be absorbed by the subject without adverse effects.

Delivery of compounds herein to wound sites may be enhanced by the use of controlled-release compositions, such as those described in PCT application WO 93/20859, which is incorporated herein by reference. Films of this type are particularly useful as coatings for prosthetic devices and surgical implants. The films may, for example, be wrapped around the outer surfaces of surgical screws, rods, pins, plates and the like. Implantable devices of this type are routinely used in orthopedic surgery. The films can also be used to coat bone filling materials, such as hydroxyapatite blocks, demineralized bone matrix plugs, collagen matrices and the like. In general, a film or device as described herein is applied to the bone at the fracture site. Application is generally by implantation into the bone or attachment to the surface using standard surgical procedures.

Biodegradable films or matrices include calcium sulfate, tricalcium phosphate, hydroxyapatite, polylactic acid, polyanhydrides, bone or dermal collagen, pure proteins, extracellular matrix components and the like and combinations thereof. Such biodegradable materials may be used in combination with non-biodegradable materials, to provide desired mechanical, cosmetic or tissue or matrix interface properties.

Alternative methods for delivery of compounds of the present invention include use of ALZET osmotic minipumps (Alza Corp., Palo Alto, Calif.); sustained release matrix materials such as those disclosed in Wang, et al. (PCT Publication WO 90/11366); electrically charged dextran beads, as disclosed in Bao, et al. (PCT Publication WO 92/03125); collagen-based delivery systems, for example, as disclosed in Ksander, et al., Ann. Surg. (1990) 211(3):288-294; methylcellulose gel systems, as disclosed in Beck, et al., J. Bone Min. Res. (1991) 6(11):1257-1265; alginate-based systems, as disclosed in Edelman, et al., Biomaterials (1991) 12:619-626 and the like. Other methods well known in the art for sustained local delivery in bone include porous coated metal prostheses that can be impregnated and solid plastic rods with therapeutic compositions incorporated within them.

Aqueous suspensions may contain the active ingredient in admixture with pharmacologically acceptable excipients, comprising suspending agents, such as methyl cellulose; and wetting agents, such as lecithin, lysolecithin or long-chain fatty alcohols. The said aqueous suspensions may also contain preservatives, coloring agents, flavoring agents, sweetening agents and the like in accordance with industry standards.

Preparations for topical and local application comprise aerosol sprays, lotions, gels and ointments in pharmaceutically appropriate vehicles which may comprise lower aliphatic alcohols, polyglycols such as glycerol, polyethylene glycol, esters of fatty acids, oils and fats, and silicones. The preparations may further comprise antioxidants, such as ascorbic acid or tocopherol, and preservatives, such as p-hydroxybenzoic acid esters.

Parenteral preparations comprise particularly sterile or sterilized products. Injectable compositions may be provided containing the active compound and any of the well known injectable carriers. These may contain salts for regulating the osmotic pressure.

If desired, the osteogenic agents can be incorporated into liposomes by any of the reported methods of preparing liposomes for use in treating various pathogenic conditions. The present compositions may utilize the compounds noted above incorporated in liposomes in order to direct these compounds to macrophages, monocytes, as well as other cells and tissues and organs, which take up the liposomal composition. The liposome-incorporated compounds of the invention can be utilized by parenteral administration, to allow for the efficacious use of lower doses of the compounds. Ligands may also be incorporated to further focus the specificity of the liposomes.

Suitable conventional methods of liposome preparation include, but are not limited to, those disclosed by Bangham, A. D., et al., J. Mol. Biol. (1965) 23:238-252, Olson, F., et al., Biochem. Biophys. Acta. (1979) 557:9-23, Szoka, F., et al., Proc. Natl. Acad. Sci. USA (1978) 75:4194-4198, Kim, S., et al., Biochem. Biophys. Acta. (1983) 728:339:348, and Mayer, et al., Biochem. Biophys. Acta. (1986) 858:161-168.

The liposomes may be made from the present compounds in combination with any of the conventional synthetic or natural phospholipid liposome materials including phospholipids from natural sources such as egg, plant or animal sources such as phosphatidylcholine, phosphatidylethanolamine, phosphatidylglycerol, sphingomyelin, phosphatidylserine, or phosphatidylinositol and the like. Synthetic phospholipids that may also be used, include, but are not limited to: dimyristoylphosphatidylcholine, dioleoylphosphatidylcholine, dipalmitoylphosphatidylcholine and distearoylphosphatidycholine, and the corresponding synthetic phosphatidylethanolamines and phosphatidylglycerols. Cholesterol or other sterols, cholesterol hemisuccinate, glycolipids, cerebrosides, fatty acids, gangliosides, sphingolipids, 1,2-bis(oleoyloxy)-3-(trimethyl ammonio) propane (DOTAP), N-[1-(2,3-dioleoyl) propyl-N,N,N-trimethylammonium chloride (DOTMA), and other cationic lipids may be incorporated into the liposomes, as is known to those skilled in the art. The relative amounts of phospholipid and additives used in the liposomes may be varied if desired. The preferred ranges are from about 60 to 90 mole percent of the phospholipid; cholesterol, cholesterol hemisuccinate, fatty acids or cationic lipids may be used in amounts ranging from 0 to 50 mole percent. The amounts of the present compounds incorporated into the lipid layer of liposomes can be varied with the concentration of the lipids ranging from about 0.01 to about 50 mole percent.

The liposomes with the above formulations may be made still more specific for their intended targets with the incorporation of monoclonal antibodies or other ligands specific for a target. For example, monoclonal antibodies to the BMP receptor may be incorporated into the liposome by linkage to phosphatidylethanolamine (PE) incorporated into the liposome by the method of Leserman, L., et al., Nature (1980) 288:602-604.

In addition, any of the formulations useful herein may include other active or inert components. Of particular interest are those agents that promote tissue growth or infiltration, such as growth factors. Exemplary growth factors for this purpose include epidermal growth factor (EGF), fibroblast growth factor (FGF), platelet-derived growth factor (PDGF), transforming growth factor β (TGF-β), parathyroid hormone (PTH), leukemia inhibitory factor (LIF), insulin-like growth factors (IGFs) and the like. Agents that promote bone growth, such as bone morphogenetic proteins (U.S. Pat. No. 4,761,471; PCT Publication WO 90/11366), osteogenin (Sampath, et al., Proc. Natl. Acad. Sci. USA (1987) 84:7109-7113) and NaF (Tencer, et al., J. Biomed. Mat. Res. (1989) 23: 571-589) are also contemplated.

The compounds may also be used in conjunction with agents that inhibit bone resorption. Antiresorptive agents, such as estrogen, bisphosphonates and calcitonin, are preferred for this purpose. More specifically, the compounds disclosed herein may be administered for a period of time (for instance, months to years) sufficient to obtain correction of a bone deficit condition. Once the bone deficit condition has been corrected, the vertebrate can be administered an anti-resorptive compound to maintain the corrected bone condition. Alternatively, the compounds may be administered with an anti-resorptive compound in a cyclical manner (administration of compound, followed by anti-resorptive, followed by compound, and the like).

Veterinary uses of the disclosed compounds are also contemplated. Such uses would include treatment of bone or cartilage deficits or defects, i.e., bone disorders, in domestic animals, livestock and thoroughbred horses.

The compounds identified by the method of the present invention may be used to stimulate growth of bone-forming cells or their precursors, or to induce differentiation of bone-forming cell precursors, either in vitro or ex vivo. The compounds may also modify a target tissue or organ environment, so as to attract bone-forming cells to an environment in need of such cells. As used herein, the term “precursor cell” refers to a cell that is committed to a differentiation pathway, but that generally does not express markers or function as a mature, fully differentiated cell. As used herein, the term “mesenchymal cells” or “mesenchymal stem cells” refers to pluripotent progenitor cells that are capable of dividing many times, and whose progeny will give rise to skeletal tissues, including cartilage, bone, tendon, ligament, marrow stroma and connective tissue (see Caplan, A., J. Orthop. Res. (1991) 9:641-650). As used herein, the term “osteogenic cells” includes osteoblasts and osteoblast precursor cells. More particularly, the compounds are useful for stimulating a cell population containing marrow mesenchymal cells, thereby increasing the number of osteogenic cells in that cell population. In a preferred method, hematopoietic cells are removed from the cell population, either before or after stimulation with the disclosed compounds. Through practice of such methods, osteogenic cells may be expanded. The expanded osteogenic cells can be infused (or reinfused) into a vertebrate subject in need thereof. For instance, a subject's own mesenchymal stem cells can be exposed to compounds of the present invention ex vivo, and the resultant osteogenic cells could be infused or directed to a desired site within the subject, where further proliferation and/or differentiation of the osteogenic cells can occur without immunorejection. Alternatively, the cell population exposed to the compounds may be immortalized human fetal osteoblastic or osteogenic cells. If such cells are infused or implanted in a vertebrate subject, it may be advantageous to “immunoprotect” these non-self cells, or to immunosuppress (preferably locally) the recipient to enhance transplantation and bone or cartilage repair.

An “effective amount” of a composition is that amount which produces a statistically significant effect. For example, an “effective amount” for therapeutic uses is the amount of the composition comprising an active compound herein required to provide a clinically significant increase in healing rates in fracture repair; reversal of bone loss in osteoporosis; reversal of cartilage defects or disorders; prevention or delay of onset of osteoporosis; stimulation and/or augmentation of bone formation in fracture non-unions and distraction osteogenesis; increase and/or acceleration of bone growth into prosthetic devices; and repair of dental defects. Such effective amounts will be determined using routine optimization techniques and are dependent on the particular condition to be treated, the condition of the patient, the route of administration, the formulation, and the judgment of the practitioner and other factors evident to those skilled in the art. The dosage required for the compounds of the invention (for example, in osteoporosis where an increase in bone formation is desired) is manifested as a statistically significant difference in bone mass between treatment and control groups. This difference in bone mass may be seen, for example, as a 5-20% or more increase in bone mass in the treatment group. Other measurements of clinically significant increases in healing may include, for example, tests for breaking strength and tension, breaking strength and torsion, 4-point bending, increased connectivity in bone biopsies and other biomechanical tests well known to those skilled in the art. General guidance for treatment regimens is obtained from experiments carried out in animal models of the disease of interest.

The dosage of the compounds will vary according to the extent and severity of the need for treatment, the activity of the administered compound, the general health of the subject, and other considerations well known to the skilled artisan. Generally, they can be administered to a typical human on a daily basis as an oral dose of about 0.1 mg/kg-1000 mg/kg, and more preferably from about 1 mg/kg to about 200 mg/kg. The parenteral dose will appropriately be 20-100% of the oral dose. While oral administration may be preferable in most instances (for reasons of ease, patient acceptability, and the like), alternative methods of administration may be appropriate or required for selected compounds and selected defects or diseases.

The following examples are intended to illustrate but not to limit the invention.

### EXAMPLE 1

### Verification of the Nexus Between Proteasome Inhibition and BMP Expression and Function

A. Proteasome Inhibitors Enhance BMP-2 Expression

2T3 murine osteoblast precursor cells and MG-63 human osteoblastic cells were cultured with α minimal essential medium (αMEM) supplemented with 10% fetal calf serum (FCS).

2T3 cells were stably transfected with murine BMP-2 promoter (−2712/+165) linked to firefly luciferase cDNA (Ghosh-Choudhury, et al., Endocrinol. (1996) 137:331-339). The cells were plated in 96-well culture plates and cultured for 24 h. The cells were then treated with different proteasome inhibitors or other compounds for additional 24 h. The cell lysates were extracted and luciferase activities were measured by a luciferase assay kit (Promega, Madison, Wis.) using a Luminometer.

The natural product epoxomicin and the peptide aldehyde proteasome inhibitor 1 (PS-1) both increased luciferase activity in 2T3 cells stably transfected with the murine BMP-2 promoter (−2712/+165) operatively linked to the firefly luciferase cDNA. These compounds were considerably more potent as stimulators of BMP-2 expression than the statins, which inhibit the enzyme hydroxymethyl glutaryl (HMG)-CoA reductase, and have recently been shown to stimulate BMP-2 expression and enhance bone formation in vitro and in vivo (Mundy, et al., Science, (1999) 286:1946-1949).

To determine the effects of proteasome inhibitors on BMP-2 mRNA expression, Northern blot analysis was performed. MG-63 cells were plated in T-150 flasks at density of 2×10⁶ cells per flask for RNA extraction. Total RNA was extracted from MG-63 cells using RNAzol B method (Tel-test Inc., Houston, Tex.). Enrichment of polyadenylated RNA was obtained using Stratagene oligo(dT) cellulose columns (Stratagene, San Diego, Calif.). 5 μg of poly (A⁺) RNA was denatured in 2.2 M formaldehyde and 50% fornamide and run on an 1% agarose gel containing 2.2 M formaldehyde. The gels were transferred to a Nytran filter (Schleicher and Schuell, Keene, N.H.) by capillary blotting with 20× SSC (1× SSC=150 mM NaCl, 15 mM Na Citrate, pH 7.0). The RNA was then cross-linked to the filter by UV irradiation (Stratalink, Stratagene, San Diego, Calif.). Prehybridization was carried out at 56° C. in 5× SSC containing 50% formamide and 150 μg/ml of denatured salmon sperm DNA. After 1-2 h prehybridization, the DNA probes were added to the hybridization solution at a concentration of 5×10⁵ cpm/ml. Hybridization was carried out for 15 h at 56° C. The filters were then washed twice with 2× SSC and 0.1% SDS, once with 0.5% SSC and 0.1% SDS, and once with 0.1× SSC and 0.1% SDS at 56° C. for 15 min each. The filters were dried at room temperature, exposed at −70° C. overnight and then quantitated in cpm using an AMBIS Image Acquisition and Analysis System (AMBIS, San Diego, Calif.).

MG-63 cells were plated in 12-well culture plates at the density of 2.5×10⁵ cells/well and cultured with αMEM supplemented with 10% FCS for 24 h. The cells were treated with the proteasome inhibitors epoxomicin and PSI for 24 h. After incubation, the cell lysates were extracted with 150 μl lysis buffer (50 mM TrisHCl pH 8.0, 150 mM NaCl, 1% NP40) with protease inhibitors. The lysates were used to measure the BMP-2 protein production by ELISA.

For protein production, 96-well Dynatech white plates were coated with mouse anti-hBMP-2 monoclonal antibody (raised against rhBMP-2) at 1 μg/ml in PBS (100 μl/well) and incubated overnight at 4° C. The following procedures were performed at room temperature. The plates were washed 3 times with 300 μl of PBS-T wash buffer (PBS and 0.05% Tween) and then blocked with 300 μl 0.2% I-Block solution (0.2% I-Block in PBS-T, Applied Biosystems) for 2 h. 100 μl cell lysates were then added and incubated with coated antibody for 2 h. The plates were then washed with wash buffer 3 times and incubated with 100 μl detection antibody (goat anti-human BMP-2 polyclonal antibody, 1 μg/ml, 1:200 in PBS-T/2% FCS, Santa Cruz) for 2 h. The plates were then washed with wash buffer 3 times and incubated with 100 μl alkaline phosphatase-conjugated mouse anti-goat IgG (Sigma, 1:50,000 in PBS-T/2%FCS) for 1 h. After 3 time washing with wash buffer and 3 time washing with assay buffer, the plates were incubated with 100 μl Tropix enzyme substrate (Applied Biosystems) in dark for 10 min and then read light emission with a Luminometer. rhBMP-2 (R&D) was used to generate the standard curve.

The results showed that the proteasome inhibitors tested above (epoxomicin and PS-1) also increased BMP-2 mRNA and BMP-2 protein expression in cultured human MG-63 osteoblastic cells, but had no effect on expression of BMP-4 mRNA.

B. Effect of Noggin on Bone Formation Induced by Proteasome Inhibitors

Organ cultures of neonatal murine calvarial bones were used as described by Traianedes, K., et al., Endocrinology (1998) 139:3178-3184. Bones were removed from the calvaria of 4-5 day old ICR Swiss mice and cultured in BGJ medium with 0.1% BSA for 72 hours together with test compounds. Mouse noggin/FC chimera was purchased from R&D Systems, Stillwater, Minn., and added at 2 μg/ml. This is a high affinity binding protein for BMP-2, BMP-4 and BMP-7.

The addition of noggin inhibited the bone stimulating effects of the proteasome inhibitors PS-1 and epoxomicin and also the bone stimulating effect of BMP-2 in these cultures. Noggin had no effect on bone formation stimulated by acidic fibroblast growth factor (aFGF), however. Thus, the effect of the proteasome inhibitors on bone formation is further correlated with their effects on BMP-2 expression.

### EXAMPLE 2

### Control of BMP-2 Expression by Gli3 Processing in Osteoblasts

A. Epoxomicin and PS-1 Inhibit Gli3 Degradation

Full-length human Gli3 expression plasmid (pact-Flag-Gli3) was kindly provided by Dr. Shunsuke Ishii (Dai, P., et al., J. Biol. Chem. (1999) 274:8143-8152). The molecular weights for expressed Gli3 proteins by transfection of full-length Gli3 cDNA is about 190 kd (Gli3¹⁹⁰) and the processed form of Gli3 is about 85 kd (Gli3⁸⁵).

C3H10T1/2 multipotent progenitor cells were cultured with RPMI 1604 medium. The medium was purchased from Sigma (St. Louis, Mo.) and supplemented with 10% fetal calf serum (FCS). C3H10T1/2 cells were plated in 6-well culture plates at the density of 2×10⁵ cells/well and transfected with Flag-Gli3 (2 μg/well) and treated with IBMX (200 μM) in the presence or absence of 10 and 20 nM of epoxomicin. Gli3¹⁹⁰ protein expression was detected by Western blot using anti-Flag antibody.

293 cells were plated in 6-well culture plates and cultured with Dulbecco's modified Eagle's medium (DMEM, Sigma). 24 hours after plating, the cells were transiently transfected with Flag-Gli3 cDNA (1 μg/well) and treated with 3-isobutyl-1-methylxanthine (IBMX, 200 μM) in the presence or absence of 1, 10 and 100 nM epoxomicin or PS-1. The steady-state protein levels of Gli3 precursor protein (Gli3¹⁹⁰) or processed form of Gli3 (Gli3⁸⁵) were detected by Western blot using anti-Flag antibody.

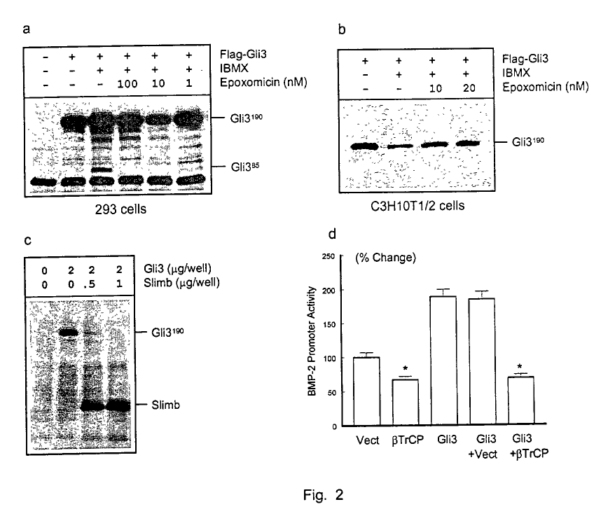

In 293 cells, both PS-1 and epoxomicin impaired Gli3 processing to its processed shorter form. As shown by the gel displayed in FIG. 2a, the band represented by the short form Gli3⁸⁵ is abolished in the presence of as little as 10 nM epoxomicin, and substantially reduced even at 1 nM epoxomicin. In C3H10T1/2 cells, PS-1 and epoxomicin prevent the degradation of Gli3 precursor protein (Gli3¹⁹⁰). As shown in FIG. 2b, epoxomicin also inhibits degradation of the precursor form of Gli3, Gli3¹⁹⁰ in these cells, as shown by intensity of the bands. (Gli3⁸⁵ runs off the gel.)

B. Production of β-TrCP Reduces Protein levels of the Precursor Form of Gli3

Murine β-TrCP cDNA (FWD1) was amplified by RT-PCR using template RNA extracted from 2T3 cells and then cloned into p3×Flag-CMV vector (Sigma, St. Louis, Mo.). The nucleotide sequence was verified by sequencing the entire cDNA. C3H10T1/2 cells were plated in 6-well culture plates and Flag-Gli3 (2 μg/well) and β-TrCP (0.5 and 1 μg/well) were co-transfected in C3H10T1/2 cells. 48 h after transfection, cell lysates were extracted and Gli3¹⁹⁰ protein expression was detected by Western blot using anti-Flag antibody.

The levels of Gli3 precursor protein (Gli3¹⁹⁰) were reduced when C3H10T1/2 cells were co-transfected with β-TrCP. This is shown in FIG. 2c; in this figure, the presence of β-TrCP is labeled “Slimb,” the former name for this enzyme.

C. Production of Gli3 Enhances BMP-2 Promoter Activity and β-TrCP Reduces BMP-2 Promoter Activity

C2C12 cells were plated in 24-well culture plates at density of 2×10⁴ cells/well. Murine BMP-2 promoter (−2712/+165)-luciferase reporter construct was co-transfected with full-length Gli3 (100 ng/well), or with β-TrCP (100 ng/well) or with full-length Gli3 (100 ng/well) plus β-TrCP (100 ng/well) expression plasmids into C2C12 cells. 24 h after transfection, the cell lysates were extracted and luciferase activities were measured using Luminometer. Transfection with β-TrCP reduced basal levels of BMP-2 promoter activity and blocked Gli3-induced BMP-2 promoter activity. As shown in FIG. 2d, the presence of β-TrCP reduces BMP-2 promoter activity in the presence and absence of Gli3; the presence of Gli3 precursor enhances BMP-2 promoter activity as expected.

### EXAMPLE 3

### Effect of Full-Length and Truncated Gli3 on BMP-2 Promoter Activity and mRNA Expression

A. BMP-2 Promoter Assay

The N-terminal truncated form of Gli3 cDNA (trGli3) encoding amino acid 1-748 was derived from the full-length Gli3 cDNA (pact-Flag-Gli3). The pact-Flag-Gli3 plasmid was first digested with ClaI/XbaI restriction enzymes to remove the nucleotide sequence encoding C-terminal part of the Gli3 and a polylinker containing the ClaI site, stop codon and XbaI site was cloned into the ClaI/XbaI sites of the Gli3 plasmid, in which the nucleotide sequence encoding C-terminal part of the Gli3 was removed.

C2C12 myoblast/osteoblast precursor cells were plated in 24-well culture plates at density of 2×10⁴ cells/well. Murine BMP-2 promoter (−2712/+165)-luciferase reporter construct was co-transfected with different amounts (62.5, 125 and 250 ng/well) of full-length Gli3 cDNA or truncated Gli3 cDNA. 48 h after transfection, the cell lysates were extracted and luciferase activity was measured using a Luminometer.

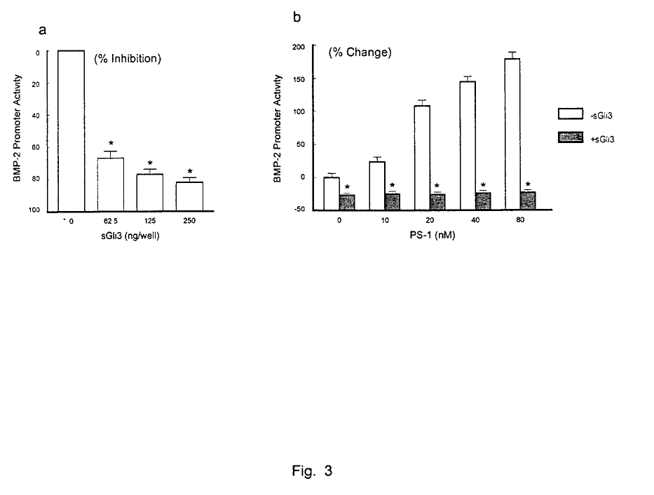

The BMP-2 promoter luciferase assay measured the effects of full-length and truncated Gli3 on the promoter activity of BMP-2 gene. The results show that the full-length Gli3 slightly enhanced BMP promoter activity, while the truncated Gli3 strongly suppressed it. FIG. 3a shows only the results for the shortened protein (sGli3).

B. BMP-2 mRNA Assay

Full-length Gli3 cDNA (250 ng/well) was transfected into C2C12 cells and BMP-2 mRNA expression was detected by RT-PCR. 48 h after transfection, the incubation was stopped and total RNA was extracted from these cells. First strand cDNA was synthesized from 10 μg of total RNA with an 18-mer oligo dT primer using Superscript reverse transcriptase (Gibco/BRL Life Technologies). The cDNA was then used as the template and the PCR was performed using SuperMix (Gibco/BRL Life Technologies). The results showed that full-length Gli3 enhanced the production of BMP-2 mRNA.

C. Effect of Truncated Gli3 on PS-1-induced BMP-2 Promoter

Effects of truncated Gli3 (100 ng/well) on PS-1-induced BMP-2 promoter activity were measured in C2C12 cells using the procedure of Example 2C. The concentrations of PS-1 used in this experiment are 10-80 nM. It was found that transfection of C2C12 cells with an expression plasmid of truncated Gli3 completely blocked the effect of PS-1 on the BMP promoter activity (FIG. 3b). Typically, PS-1 enhances BMP-promoter activity; it is incapable of doing so when increasing amounts of sGli3 are added in a dose-dependent manner. This confirms that the effect of the proteasome inhibitor PS-1 can be explained as an effect on Gli3 processing.

### EXAMPLE 4

### Effect of trGli3 on Bone Formation

Neonatal murine calvariae were transfected with either empty vector or truncated Gli3 (trGli3) using lipofectamine plus reagents for the first 24 hours and then treated with epoxomicin (20 nM) for the remaining 3 days of the assay according to Traianedes, et al., 1998 (supra). In two separate experiments, transfection of trGli3 impaired markedly the effects of epoxomicin on osteoblast differentiation and bone formation, but had no effect on bone formation stimulated by the unrelated anabolic agent aFGF as shown by histology. These data further support the biologic importance of Gli3 processing by the ubiquitin-proteasome pathway in osteoblasts.

### EXAMPLE 5

### Induction of Alkaline Phosphatase (ALP) Activity and Osteocalcin Production by Smad1 and Mutant Smurf1

C2C12 cells were plated in 24-well culture plates at density of 2×10⁴ cells/well and incubated with DMEM supplemented with 10% FCS. 24 h later, C2C12 cells were transfected with empty vector, Smad1 or mutant Smurf1 expression plasmid using lipofactamine plus reagents (Gibco/BRL). Recombinant human BMP-2 (100 ng/ml) (R & D) was added next day and the cells were incubated for additional 24 hours. At the end of incubation, the medium was collected for osteocalcin assay and the cell lysates were extracted using 200 μl 0.05% Triton X-100 (freeze-thaw 3 times) and the ALP activity was measured using a Sigma ALP assay kit (Sigma Chemical co., St Louis) and protein content was measured using Bio-Rad protein assay reagents. The ALP activity was calculated using these two parameters.

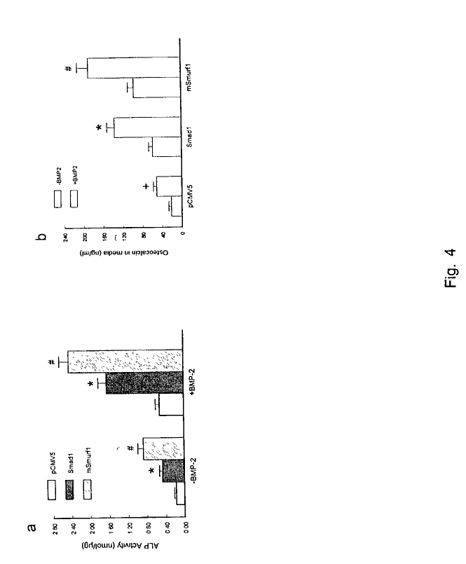

Addition of BMP-2 (100 ng/ml) increased ALP activity (FIG. 4a) and osteocalcin production (FIG. 4b) in C2C12 cells. Transfection of wild-type Smad1 and mutant Smurf1 (C710A) expression plasmids (mutant Smurf1 lacks catalytic activity) into C2C12 cells increased basal levels as well as BMP-2-induced ALP activity and osteocalcin production. Alkaline phosphatase activity and osteocalcin activity are downstream of BMP-2 signaling; thus, the addition of BMP-2 generically enhances the levels of these proteins. As Smad1 is instrumental in this signaling, its presence, as shown, enhances the levels of these proteins over control vector, both in the presence and absence of BMP-2. As the mutant Smurf1 lacks catalytic activity, it acts as a decoy and inhibits the degradation of endogenous Smad1 by endogenous Smurf1; thus, the presence of this protein further enhances the levels of these proteins both in the presence and absence of BMP-2.

### EXAMPLE 6

### Smurf1 Reduces Steady State Levels of Smad1

The effects of proteasome inhibitors on steady-state levels of Smad1 protein were examined in C2C12 cells. C2C12 cells were plated at 6-well culture plates at density of 2×10⁵ cells/well and cultured with αMEM supplemented with 10% FCS for 24 h. Flag-Smad1 expression plasmid was transfected in C2C12 cells. 24 h later, the cell lysates were extracted and Smad1 protein expression was detected by Western blot using anti-Flag antibody.

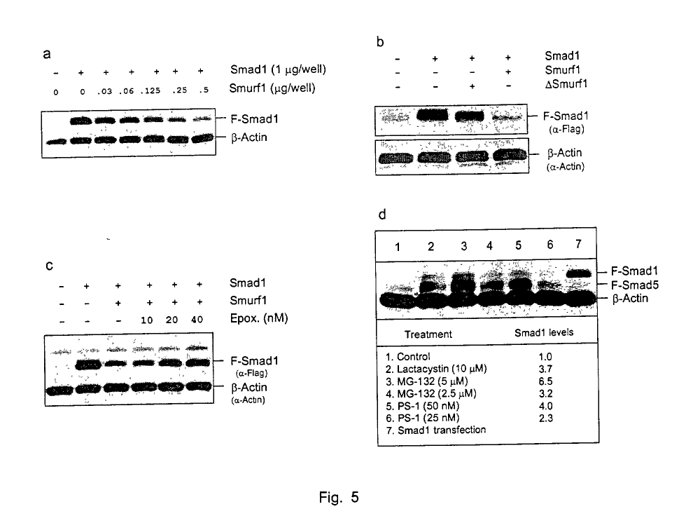

Different amounts of Smurf1 expression plasmid (0.03-0.5 μg/well) were co-transfected with Flag-Smad1 expression plasmid in C2C12 cells. Smad1 protein expression was detected by Western blot. Expression of Smurf1 decreased steady-state levels of Smad1 in a dose-dependent manner (FIG. 5a). Co-transfection of mutant Smurf1 (C710A) slightly reduced Smad1 protein level (FIG. 5b). Actin was used as a standard to demonstrate that there was no non-specific effect of transfection on production of Smad1.

### EXAMPLE 7

### Proteasome Inhibitors Prevent Smad1 Degradation

Effects of proteasome inhibitors on steady-state levels of Smad1 protein were examined in C2C12 cells. C2C12 cells were plated in 6-well culture plates at density of 2×10⁵ cells/well and cultured with DMEM supplemented with 10% FCS for 24 h. The cells were then transfected with Smad1 and Smurf1 expression plasmids. 24 h after transfection, the cells were treated with different proteasome inhibitors at different concentrations and incubated for additional 24 h.

Treatment of C2C12 cells with MG-132 (5 μM), lactacystin (10 μM), PS-1 (25 and 50 nM) and epoxomicin (10, 20 and 40 nM) partially or completely reversed Smurf1-induced Smad1 degradation. The results with respect to epoxomicin are shown in FIG. 5c.

### EXAMPLE 8

### Proteasome Inhibitors Increase Levels of Endogenous Smad1

To determine the effects of proteasome inhibitors on the steady-state levels of endogenous Smad1 protein, C2C12 cells were plated in 6-well culture plates (2×10⁵ cells/well) and cultured with DMEM supplemented with 10% FCS. 24 h later, proteasome inhibitors lactacystin (10 μM), MG-132 (2.5 and 5 μM), PS-1 (25 and 50 nM) were added into C2C12 cells. The cells were cultured with the proteasome inhibitors for 24 hours and then the cell lysates were extracted. The steady-state levels of endogenous Smad1 were analyzed by Western blot using Smad1 antibody (Santa Cruz).

Treatment of proteasome inhibitors in C2C12 cells increased endogenous Smad1 protein levels. The maximal effects of the proteasome inhibitors on Smad1 were reached when C2C12 cells were treated with MG-132 (5 μM, 6.5-fold increase). As shown in FIG. 5d, as compared to control, Smad1 levels were enhanced by PS-1 and lactacystin as well. Thus, lactacystin at 10 μM enhanced the level 3.7-fold; MG-132 at 5 μM enhanced the level 6.5-fold; PS-1 at 50 nM enhanced the level 4.0-fold.

### EXAMPLE 9

### Smurf1 Mediates Cbfa1 Degradation

A. Smurf1 Mediates Cbfa1 Degradation

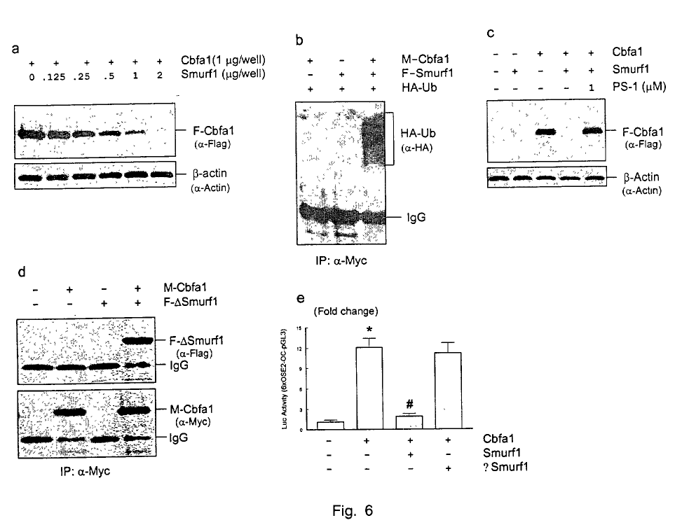

Since Cbfa1 interacts with Smad1, whose degradation is mediated by Smurf1, the effect of Smurf1 on Cbfa1 degradation in myoblast/osteoblast precursor C2C12 cells and osteoblast precursor 2T3 cells was tested. Flag-tagged Cbfa1 expression plasmid was co-transfected with different amounts of Smurf1 expression plasmid into C2C12 and 2T3 cells. Expression of Smurf1 greatly reduced steady-state levels of Cbfa1 in a dose-dependent manner (FIG. 6a). Expression of an unrelated Hect domain E3 ligase, Itch, with Cbfa1 had no effect on the degradation of Cbfa1 (data not shown), suggesting that Smurf1-mediated Cbfa1 degradation is a specific effect of the ligase.

B. Smurf1-Mediated Cbfa1 Degradation is Ubiquitin-Proteasome-Dependent

Smurf1, Cbfa1 and ubiquitin expression plasmids were cotransfected into C2C12 cells. After immunoprecipitating the Cbfa1 protein, a typical ubiquitinated Cbfa1 protein ladder was observed (FIG. 6b), indicating that Smurf1-mediated Cbfa1 degradation is ubiquitin-dependent. The above described C2C12 cells were treated with proteasome inhibitor 1 (PS-1, 1 μM) for 2 h after transfection the total degradation of Cbfa1 effected by Smurf1 was completely abolished by treatment with PS-1 (FIG. 6c). Similar results were also obtained for epoxomicin (data not shown). These results demonstrate that Smurf1-mediated Cbfa1 degradation is proteasome-dependent.

C. Smurf1 Physically Interacts with Cbfa1

Cbfa1 and mutant Smurf1 (ΔSmurf1, C710A) expression plasmids were co-transfected into C2C12 cells. As Smurf1 mediates the degradation of proteins with which it interacts, the protein complex was stabilized by using an expression plasmid for the catalytic point mutant of Smurf1 (ΔSmurf1) as described by Zhu, et al., Nature (1999) 400:687-693 (supra). Using epitope-tagged proteins in co-immunoprecipitation and Western blot analyses, it was found that Cbfa1 co-precipitates with ΔSmurf1 (FIG. 6d). A putative PY motif, presumably bound with the WW domain of Smurf1 protein, was found at the C-terminus of Cbfa1 protein. These results suggest that Smurf1 interacts with Cbfa1 and mediates its degradation.

D. Smurf1 Modulates Cbfa1 Function

The ability of Cbfa1 to activate the reporter gene, 6×OSE2-OC-Luc (Ducy and Karsenty, 1995), in C2C12 cells was tested. This represents the activity of the osteocalcin basal promoter. A Cbfa1 expression plasmid and 6×OSE2-OC-Luc reporter construct which has 6 copies of the Cbfa1. Transcription target sequences were co-transfected into C2C12 cells in the presence and absence of Smurf1 or ΔSmurf1. Expression of Cbfa1 significantly increased activity of 6×OSE2-OC-Luc reporter. Co-transfection of Smurf1 with Cbfa1 significantly reduced Cbfa1 -induced luciferase activity of this reporter gene. In contrast, co-transfection of ΔSmurf1 lacking in catalytic activity, had no significant effect on Cbfa1-induced luciferase activity of the reporter gene (FIG. 6e). These results demonstrate that Smurf1 mediates Cbfa1 degradation and leads to a decrease in Cbfa1 activity.

1. A method to identify a compound or protocol which enhances bone formation and/or osteoblast differentiation which method comprises assessing the ability of a candidate compound or protocol to inhibit the activity of β-TrCP, whereby a compound or protocol which inhibits this activity is identified as a compound or protocol which will enhance bone formation and/or osteoblast differentiation.

2. A method to identify a compound or protocol which enhances bone formation and/or osteoblast differentiation which method comprises assessing the ability of a candidate compound or protocol to inhibit the activity of Smurf1 or Smurf2, whereby a compound or protocol which inhibits this activity is identified as a compound or protocol which will enhance bone formation and/or osteoblast differentiation.

3. A method to enhance bone formation and/or osteoblast differentiation or proliferation by administering to a subject in need of such treatment compounds or protocols identified by the method of claim 1.

4. A method to enhance bone formation and/or osteoblast differentiation or proliferation by administering to a subject in need of such treatment compounds or protocols identified by the method of claim 2.

5. A method to treat bone defect conditions which method comprises administering to a subject in need of such treatment an effective amount of a protocol or compound which is inhibitory to β-TrCP.

6. A method to treat bone defect conditions which method comprises administering to a subject in need of such treatment an effective amount of a protocol or compound which is inhibitory to Smurf1.

7. A method to treat bone defect conditions which method comprises administering to a subject in need of such treatment an effective amount of a protocol or compound which is inhibitory to Smurf2.
